(19) United States
(12) Patent Application Publication
(10) Pub. No.: US 2017/0191991 A1
(43) Pub. Date: Jul. 6, 2017

(54) REUSABLE CHEMICAL OR BIOLOGICAL SENSOR AND METHOD FOR USING SAME

(71) Applicant: SEOUL NATIONAL UNIVERSITY R&DB FOUNDATION, Seoul (KR)

(72) Inventors: Seung Hun HONG, Seoul (KR); Xing CHEN, Seoul (KR); Ha Neul YOO, Seoul (KR)

(21) Appl. No.: 15/313,337

(22) PCT Filed: May 29, 2015
(86) PCT No.: PCT/KR2015/005393

§ 371 Date: Nov. 22, 2016

(30) Foreign Application Priority Data
May 29, 2014 (KR) 10-2014-0064964

(51) Int. Cl. G01N 33/53; B82Y 15/00

(52) U.S. Cl. CPC G01N 33/5302; B82Y 15/00; G01N 2446/00

(57) ABSTRACT

For a chemical or biological sensor, which is reusable while maintaining a clean state thereof, and a method for using the same, the present invention provides a reusable chemical or biological sensor and a method for using the same, the reusable chemical or biological sensor comprising: a sensor transducer; a ferromagnetic pattern formed on at least one surface of the sensor transducer; magnetic nanoparticles which can be collected or released in a single layer on the sensor transducer, in directions of first and second magnetic fields applied to the sensor transducer; and a receptor which is fixed on the magnetic nanoparticles and can bind to a target substance to be detected.

### TECHNICAL FIELD

The present invention relates to a chemical or biological sensor technology, and more particularly to a reusable chemical or biological sensor and a method for using same.

### BACKGROUND ART

In general, a chemical or biological sensor includes a signal transducer to which is fixed a receptor acting as a sensing material. The sensor has an advantage of very sensitively detecting analytes through a specific and strong interaction between the receptor and the analytes.

Receptors are materials that can be specifically bound to analytes and typical examples of the receptors include antibodies, DNAs, carbohydrates, etc. In using the above-described biosensors to detect different analytes, different sensor chips to which different receptors for different analytes are fixed must be used. Thus, it is problematic that the development of sensor chips costs a lot and the use of the chips is not easy.

Furthermore, when detecting analytes such as food or blood, in which a lot of different substances are mixed, it is problematic that interference from different substances hampers accurate detection. To solve this problem, pre-treatment methods are used, which separate analytes from other substances by using magnetic nanoparticles to which receptors are fixed.

However, when the magnetic nanoparticles are collected, the particles that are not bound to the analytes as well as the particles that are bound to the analytes are collected together, and, therefore, a sensor chip to which another receptor is fixed must be used to detect the analytes only.

In more detail, when a receptor fixed to a magnetic nanoparticle is referred to as “A”, another receptor “B” must be fixed to the sensor chip and the two receptors A and B must be bound to different parts of the analytes such that the binding of one receptor does not affect that of the other. Therefore, usually two different kinds of monoclonal antibodies should be used for the above detecting method and, therefore, it requires substantial effort and is costly. Furthermore, it is problematic that different sensor chips must be used for different analytes as described above.

### DETAILED DESCRIPTION OF THE INVENTION

### Technical Problem

The object of the present invention is to solve various problems including the above-described problems and provide a chemical or biological sensor that remains clean and is reusable and a method for using same. However, such problems are illustrative only and the scope of the present invention is not limited thereto.

### Technical Solution

According to an aspect of the present invention, a reusable chemical or biological sensor is provided. The sensor includes a sensor transducer; a ferromagnetic pattern formed on at least one surface of the sensor transducer; a magnetic nanoparticle that can be collected in a single layer on the sensor transducer or released from the sensor transducer, depending on the directions of a first magnetic field and a second magnetic field that are applied to the sensor transducer; and a receptor that is fixed to the magnetic nanoparticle and can be bound to a target substance that is to be detected.

In the reusable chemical or biological sensor, the ferromagnetic pattern may be a pattern that includes at least one of nickel and gold, and the sensor transducer includes a carbon nanotube-based sensor transducer formed on a substrate that includes at least one of silicon and silicon oxide.

In the reusable chemical or biological sensor, the ferromagnetic pattern may include a polyethylene glycol (PEG) passivation layer, and the sensor transducer may include an octadecyltrichlorosilane (OTS) passivation layer.

In the reusable chemical or biological sensor, the receptor may be an antibody and the target substance may be an antigen.

In the reusable chemical or biological sensor, the first magnetic field and the second magnetic field may be applied in directions opposite to each other to the sensor transducer.

In the reusable chemical or biological sensor, the receptor can be collected on the sensor transducer due to differences in magnetic intensity of the first magnetic field, which is caused by the ferromagnetic pattern.

In the reusable chemical or biological sensor, the receptor can be released from the sensor transducer due to differences in magnetic intensity of the second magnetic field, which is caused by the ferromagnetic pattern.

According to another aspect of the invention, method for using a reusable chemical or biological sensor is provided. The method performs at least once a unit cycle that comprises: preparing the chemical or biological sensor; collecting the magnetic nanoparticle and the receptor that is fixed to the magnetic nanoparticle on the ferromagnetic pattern by applying a first magnetic field to the chemical or biological sensor; receiving a target substance in the receptor by providing the target substance to the chemical or biological sensor; detecting the target substance by the chemical or biological sensor using an optical method or an electrical signal measurement method; and releasing the magnetic nanoparticle and the receptor by applying a second magnetic field which is in a direction opposite to the first magnetic field to the chemical or biological sensor.

### Advantageous Effects

According to one embodiment of the present invention, there are provided a chemical or biological sensor that remains clean and is reusable and a method for using same. However, the scope of the present invention is not limited thereto.

### DESCRIPTION OF THE DRAWINGS

FIG. 1 is a schematic diagram that illustrates a method of using a reusable chemical or biological sensor according to an embodiment of the present invention.

FIG. 2 is a flow chart that illustrates a method of using a reusable chemical or biological sensor according to an embodiment of the present invention.

FIG. 3 is a schematic diagram that illustrates a method of using a reusable chemical or biological sensor according to another embodiment of the present invention.

FIG. 4 shows SEM images of the magnetic nanoparticles of FIG. 2 after they are collected in a single layer on a nickel pattern and released.

FIG. 5 is a fluorescent image of the antibody that reacted to the antigen in FIG. 2.

FIG. 6 is a schematic diagram that illustrates a method of using a reusable chemical or biological sensor according to yet another embodiment of the present invention.

FIG. 7 is a graph that shows variation of current with time in the embodiment shown in FIG. 6.

FIG. 8 shows a graph that shows variation of current with log of concentration in the experimental examples of FIG. 6.

FIG. 9 is a graph that shows the degree of change in characteristics of the transistor of FIG. 6.

### BEST MODE

Hereinafter, embodiments of the present invention will be described in detail with reference to the attached drawings. The invention may, however, be embodied in many different forms and should not be construed as limited to the embodiments set forth herein. Rather, these embodiments are provided so that this disclosure will be thorough and complete, and will fully convey the scope of the invention to those skilled in the art. Furthermore, in the drawings, thicknesses and dimensions of some components may be exaggerated or reduced for clarity.

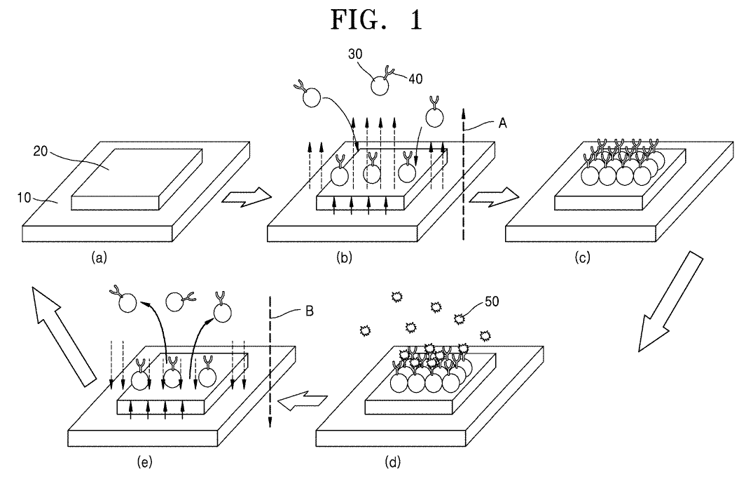

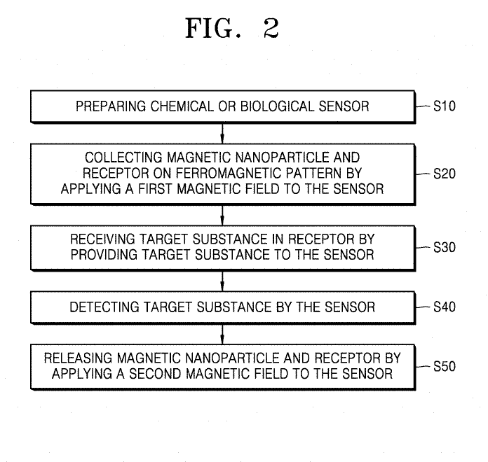

FIG. 1 is a schematic diagram that illustrates a method of using a reusable chemical or biological sensor according to an embodiment of the present invention. FIG. 2 is a flow chart that illustrates a method of using a reusable chemical or biological sensor according to an embodiment of the present invention.

Referring to FIGS. 1 and 2, a chemical or biological sensor of the present invention may include a sensor transducer 10, a ferromagnetic pattern 20, a magnetic nanoparticle 30 and a receptor 40.

For example, the ferromagnetic pattern 20 may be formed on at least one surface of the sensor transducer 10. The magnetic nanoparticle 30 may be collected in a single layer on the sensor transducer 10 or released from the sensor transducer 10 depending on the directions of a first magnetic field A and a second magnetic field B that are applied to the sensor transducer 10. The receptor 40 may be fixed to the magnetic nanoparticle 30 and may be bound to a target substance 50 that is to be detected.

Referring to FIG. 2, the reusable chemical or biological sensor according to an embodiment of the present invention may be reused by a method that performs at least once a unit cycle comprising: preparing a sensor S10; collecting a magnetic nanoparticle and a receptor in a single layer on a ferromagnetic pattern by applying a first magnetic field to the sensor S20; receiving a target substance in the receptor by providing the target substance to the sensor S30; detecting the target substance by the sensor S40; and releasing the magnetic nanoparticle and the receptor by applying a second magnetic field to the sensor S50.

For example, the chemical or biological sensor is prepared, a first magnetic field is applied to the chemical or biological sensor, and then a magnetic nanoparticle and a receptor that is fixed to the magnetic nanoparticle are collected in a single layer on the ferromagnetic pattern.

Here, when a magnetic nanoparticle and a receptor that is fixed to the magnetic nanoparticle are collected in a single layer on the ferromagnetic pattern, quantitative detection of the target substance is enabled.

Then, the target substance that is to be detected is provided to the chemical or biological sensor such that the receptor receives the target substance, and the target substance is detected by the chemical or biological sensor by an optical method or an electrical signal measurement method.

Then, a second magnetic field which is in a direction opposite to the first magnetic field is applied to the chemical or biological sensor, thereby releasing the magnetic nanoparticle and the receptor. The above-described unit cycle is performed at least once such that the chemical or biological sensor is reused.

More particularly, for example, a sensor transducer 10 on which a ferromagnetic pattern 20 is formed is placed in a solution of magnetic nanoparticles to which receptors 40 are fixed and then an external magnetic field (a first magnetic field A) is applied to the entire sensor transducer 10. Here, the ferromagnetic pattern 20 causes differences in magnetic field intensity and the magnetic nanoparticles 30 can be collected on the ferromagnetic pattern 20. Then, a sensor that includes a sensor transducer 10 to which the receptors 40 are fixed can be embodied.

Then, a target substance 50 that is to be detected is provided to the sensor such that the sensor detects the target substance 50. Here, the detection method may comprise, for example, an optical method and/or electrical signal measurement method.

After the detection is finished, a weak magnetic field (a second magnetic field B) is applied in a direction opposite to that of the previously applied magnetic field. Here, residual magnetism is left in a direction of the magnetic field (the first magnetic field A) that was applied to the ferromagnetic pattern 20, causing differences in magnetic field intensity relative to the environment. As a result, the magnetic nanoparticles 30 can be released and only the sensor transducer 10 can be left.

The present invention may repeat the above-described process, thereby repeatedly collecting and releasing the receptor 40 and providing a reusable chemical or biological sensor.

Furthermore, since no chemical substances are used and receptors 40 are collected and released effectively by a magnetic method, not only the cleanliness of the chemical or biological sensor can be maintained, but a constant detection system in which the sensor is placed in a fixed position can be provided. A miniaturized sensor can also be provided.

The sensor transducer 10 may include, for example, a carbon nanotube-based sensor transducer formed on a substrate that includes at least one of silicon and silicon oxide.

Furthermore, in order to prevent non-selective adsorption of the target substance 50 on the sensor transducer 10, the substrate portion can be passivated with, for example, octadecyltrichlorosilane (OTS).

In addition, the ferromagnetic pattern 20 is, for example, a pattern that includes at least one of nickel and gold. In order to prevent non-selective adsorption of the target substance 50 on the ferromagnetic pattern 20, the ferromagnetic pattern can be passivated using, for example, polyethylene glycol (PEG).

The magnetic nanoparticle 30 may be formed in a variety of shapes. For example, it may have a spherical shape as shown in FIG. 1, and may have a variety of shapes to which the receptor 40 can be fixed, such as a cubic, a tetrahedron, etc.

The first magnetic field A and the second magnetic field B are applied in directions opposite to each other to the sensor transducer 10. The receptor 40 can be collected on the sensor transducer 10 due to the differences in magnetic intensity of the first magnetic field A, which is caused by the ferromagnetic pattern 20. Meanwhile, the receptor 40 can be released from the sensor transducer 10 due to the differences in magnetic intensity of the second magnetic field B, which is caused by the ferromagnetic pattern 20.

However, the present invention is not limited to this, and the first magnetic field may be applied in any direction as long as it is applied to the sensor transducer 10 and the second magnetic field may be applied in any direction that is opposite to that of the first magnetic field.

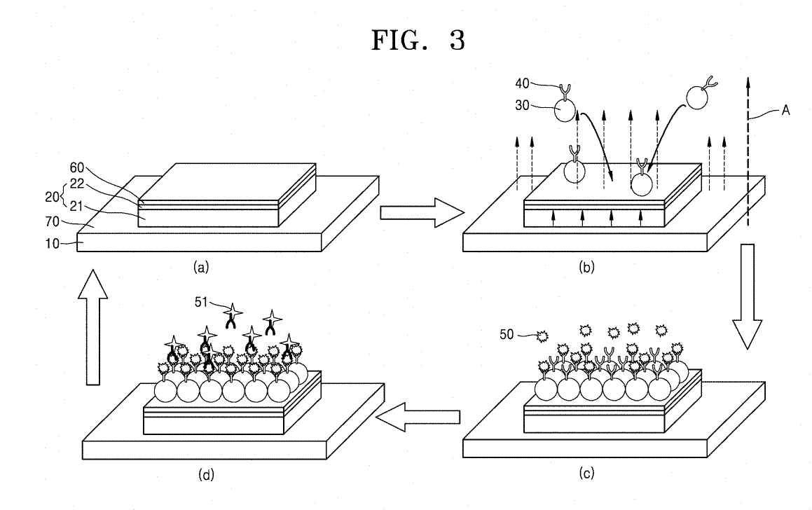

FIG. 3 is a schematic diagram that illustrates a method of using a reusable chemical or biological sensor according to another embodiment of the present invention.

Referring to FIG. 3, for example, by using a fluorescence detection method, a reusable chemical or biological sensor can be provided. Here, the receptor 40 may include an antibody and the target substance 50 may include an antigen.

Particularly, for example, as shown in (a) of FIG. 3, a sensor transducer 10 that includes a Si/SiO₂ substrates and a ferromagnetic pattern including a Ni 21/Au 22 pattern is formed on the substrate may be provided.

Then, in order to prevent non-selective adsorption of the target substance 50, the substrate portion can be passivated with octadecyltrichlorosilane (OTS) to form an OTS layer 70 and the gold Au foil may be passivated using polyethylene glycol (PEG) to form a PEG layer 60.

Then, a solution of magnetic nanoparticles 30 to which first detection antibodies (receptors 40) are fixed is prepared.

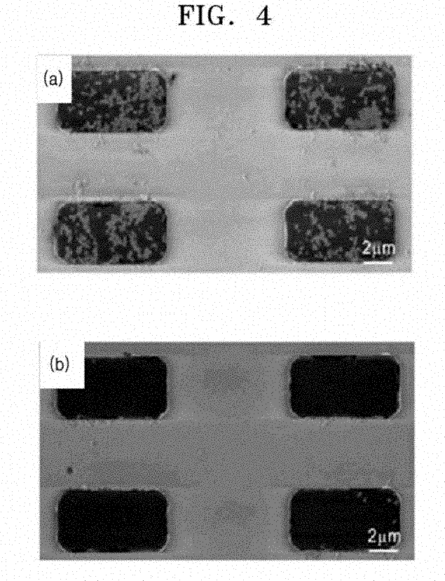

Then, as shown in (b) of FIG. 3, the sensor transducer 10 is placed in the solution of magnetic nanoparticles 30 and a first magnetic field A is applied for approximately one minute such that the magnetic nanoparticles 30 are stacked in a single layer on the ferromagnetic pattern 20. Here, (a) of FIG. 4 shows that the magnetic nanoparticles 30 of FIG. 3 are collected in a single layer on the ferromagnetic pattern 20.

Collecting magnetic nanoparticles 30 in a single layer is more advantageous than collecting in multiple layers because it enables quantitative detection. This depends on the shape and thickness of the ferromagnetic pattern, the intensity of the applied magnetic field, the duration of application of magnetic field as shown in FIG. 4. However, the present invention is not limited to this, and the magnetic nanoparticles 30 may be collected in multiple layers on the ferromagnetic pattern 20.

Then, the magnetic nanoparticles 30 that are not collected on the substrate are washed with phosphate buffered saline (PBS), and then antigens (target substances 50) that are to be detected may be provided as shown in (c) of FIG. 3.

Then, the antigens (target substances 50) that are not bound to the detection antibodies (receptors 40) are washed, and then second detection antibodies 51 that are bound to fluorescent materials are provided as shown in (d) of FIG. 3. Here, the fluorescent material may include, for example, FITC.

Next, the second detection antibodies 51 that are not bound to the antigens (target substances 50) are washed, and then the antigens can be detected using a fluorescence microscope based on fluorescence intensity.

Then, a second magnetic field B (not shown) which is in a direction opposite to that of a first magnetic field A is applied, thereby releasing the magnetic nanoparticles 30 from the sensor transducer 10. Here, (b) of FIG. 4 shows that the magnetic nanoparticles 30 of FIG. 3 are released from the ferromagnetic pattern 20.

A chemical or biological sensor which includes the above-described sensor transducer 10 from which the magnetic nanoparticles 30 have been released may be repeatedly used by performing the above-mentioned unit cycle at least once.

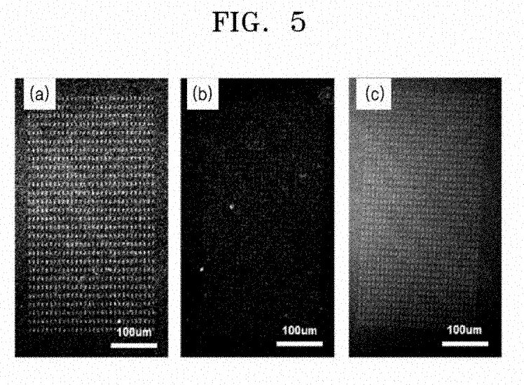

(a) of FIG. 5 shows that the antigens (target substances 50) that are bound to the first detection antibodies (receptors 40) and the second detection antibodies 51 that are bound to the antigens (target substances 50), as shown in FIG. 3, are collected on the sensor transducer 10 due to the first magnetic field. In addition, (b) of FIG. 5 shows that the antigens (target substances 50) that are bound to the first detection antibodies (receptors 40) and the second detection antibodies 51 that are bound to the antigens (target substances 50), as shown in FIG. 3, are released from the sensor transducer 10 due to the second magnetic field.

Furthermore, (c) of FIG. 5 shows that the antigens (target substances) that are bound to new, third detection antibodies (receptors) and the fourth detection antibodies (the detection antibodies bound to fluorescent materials) that are bound to the antigens (target substances) are collected on the same sensor by performing the above-described unit cycle at least once. As a result, it was confirmed that the sensor worked well even if the detection antibodies (receptors) were collected and released repeatedly.

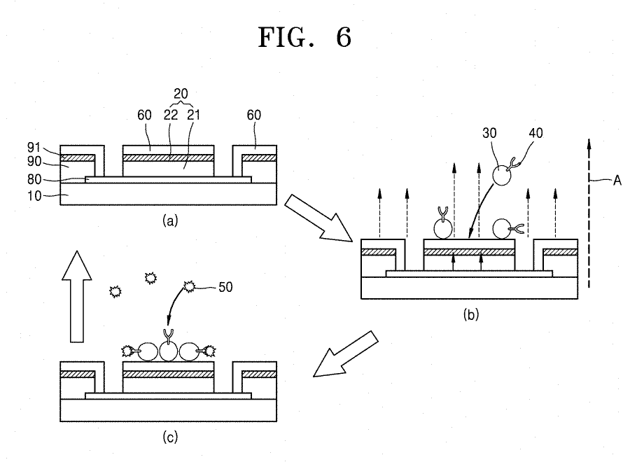

FIG. 6 is a schematic diagram that illustrates a method of using a reusable chemical or biological sensor according to yet another embodiment of the present invention.

Referring to FIG. 6, for example, by using an electrical detection method, a reusable chemical or biological sensor may be provided. Particularly, for example, as shown in (a) of FIG. 6, a carbon nanotube-based sensor transducer may be provided, in which a single-walled carbon nanotube (SWCNT) channel 80 is formed on a Si/SiO₂ substrate and a ferromagnetic pattern including a Ni 21/Au 22 pattern is formed on the SWCNT channel 80. In addition, a source and a drain on which a Ti 90/Au 91 pattern is respectively formed may be disposed on either side of the ferromagnetic pattern 20.

Then, in order to prevent non-selective adsorption of the target substance 50, the sensor transducer 10 may be passivated with PEG to form a PEG layer 60.

Then, a solution of magnetic nanoparticles 30 to which antibodies (receptors 40) are fixed is prepared.

Then, as shown in (b) of FIG. 6, the sensor transducer 10 is placed in the solution of magnetic nanoparticles 30 and a first magnetic field A is applied such that the magnetic nanoparticles 30 are collected on the ferromagnetic pattern 20.

Then, the magnetic nanoparticles 30 that are not collected on the ferromagnetic pattern 20 are washed, and then the antigens (target substances 50) that are to be detected are provided as shown in (c) of FIG. 6, thereby binding the antigens (target substances 50) to the antibodies (receptors 40).

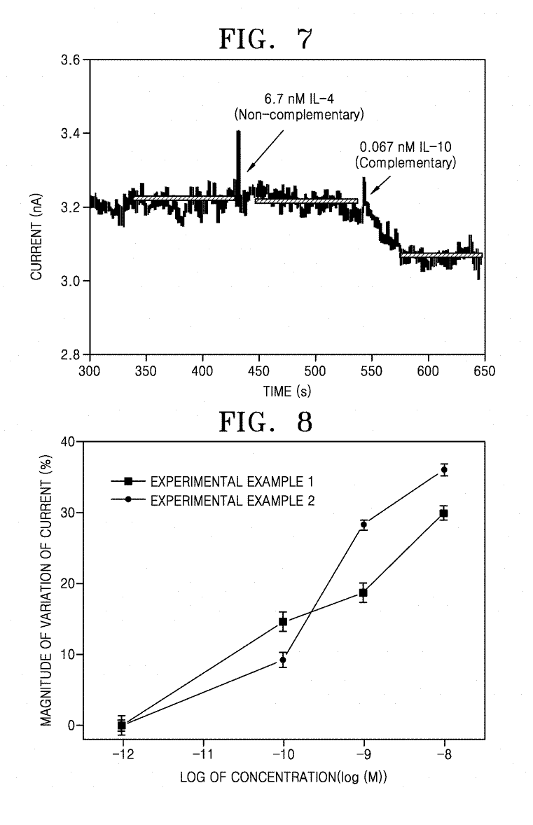

Then, as the antibody (receptor 40) is bound to the antigen (target substance 50), the work function of the ferromagnetic pattern 20 can be changed. This changes the current between the source and the drain such that the antigen (target substance 50) can be selectively detected. Here, referring to FIG. 7, the antibody (receptor 40) that was collected on the sensor transducer 10 was selectively bound to the antigen (target substance 50) at 550 seconds and since then the current decreased gradually until it became constant at about 575 seconds.

In addition, after the antigen (target substance 50) is detected, a second magnetic field B (not shown) which is in a direction opposite to a first magnetic field A is weakly applied to the sensor transducer 10, thereby releasing the magnetic nanoparticle 30 from the sensor transducer 10.

A chemical or biological sensor which includes the above-described sensor transducer 10 from which the magnetic nanoparticles 30 have been released may be used repeatedly by performing the above-mentioned unit cycle at least once.

FIG. 8 shows a graph that shows variation of current with log of concentration in the experimental examples of FIG. 6.

FIG. 8 shows that the variation of current with log of concentration when, after Human IL-4 was detected by the above-described method using a chemical or biological sensor (Experimental example 1), Human IL-10 was detected using the same chemical or biological sensor (Example 2). In each of Experimental Examples 1 and 2, a graph was plotted to show that the magnitude of variation of current increased as the log concentration increased, which shows that the sensor worked well even if it was reused repeatedly.

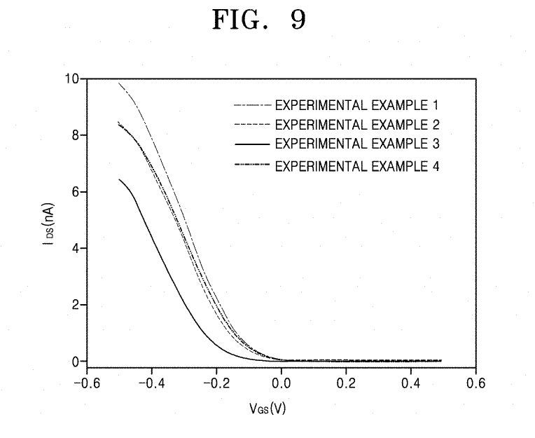

FIG. 9 is a graph that shows the degree of change in characteristics of the transistor of FIG. 6.

Referring to FIG. 9, 1 denotes a carbon nanotube-based sensor transducer ((a) of FIG. 6), 2 shows that magnetic nanoparticles are collected on a ferromagnetic pattern ((b) of FIG. 6). Furthermore, 3 shows that antibodies and antigens are bound and detected, and 4 shows that the magnetic nanoparticles 30 are released from the sensor transducer 10.

The characteristics of 4, which removes nanoparticles from 3, shows that a characteristic curve was restored to be similar to the curve of 1. This confirms that the nanoparticles were removed effectively and further confirms that the sensor could be reused.

While the present invention has been particularly shown and described with reference to embodiments thereof, it will be understood by one of ordinary skill in the art that various changes in form and details may be made therein without departing from the spirit and scope of the present invention as defined by the following claims.

1. A reusable chemical or biological sensor comprising:
a sensor transducer;
a ferromagnetic pattern formed on at least one surface of the sensor transducer;
a magnetic nanoparticle that can be collected in a single layer on the sensor transducer or released from the sensor transducer, depending on the directions of a first magnetic field and a second magnetic field that are applied to the sensor transducer; and
a receptor that is fixed to the magnetic nanoparticle and can be bound to a target substance that is to be detected.

2. The sensor of claim 1,
wherein the ferromagnetic pattern is a pattern that includes at least one of nickel and gold, and
wherein the sensor transducer includes a carbon nanotube-based sensor transducer formed on a substrate that includes at least one of silicon and silicon oxide.

3. The sensor of claim 2,
wherein the ferromagnetic pattern includes a polyethylene glycol (PEG) passivation layer, and the sensor transducer includes an octadecyltrichlorosilane (OTS) passivation layer.

4. The sensor of claim 2,
wherein the receptor is an antibody and the target substance is an antigen.

5. The sensor of claim 1,
wherein the first magnetic field and the second magnetic field are applied in directions opposite to each other to the sensor transducer.

6. The sensor of claim 5,
wherein the receptor can be collected on the sensor transducer due to differences in magnetic intensity of the first magnetic field, which is caused by the ferromagnetic pattern.

7. The sensor of claim 5,
wherein the receptor can be released from the sensor transducer due to differences in magnetic intensity of the second magnetic field, which is caused by the ferromagnetic pattern.

8. A method for using a reusable chemical or biological sensor, the method performing at least once a unit cycle that comprises:
preparing the chemical or biological sensor of claim 1;
collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

9. A method for using a reusable chemical or biological sensor, the method performing at least once a unit cycle that comprises:
preparing the chemical or biological sensor of claim 2; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

10. A method for using a reusable chemical or biological sensor, the method performing at least once a unit cycle that comprises:
preparing the chemical or biological sensor of claim 3; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

11. A method for using a reusable chemical or biological sensor, the method performing at least once a unit cycle that comprises:
preparing the chemical or biological sensor of claim 4; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

12. A method for using a reusable chemical or biological sensor, the method performing at least once a unit cycle that comprises:
preparing the chemical or biological sensor of claim 5; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

13. A method for using a reusable chemical or biological sensor, the method performing at least once a unit cycle that comprises:
preparing the chemical or biological sensor of claim 6; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.

14. A method for using a reusable chemical or biological sensor, the method performing at least once a unit cycle that comprises:
preparing the chemical or biological sensor of claim 7; collecting the magnetic nanoparticle and the receptor that is fixed to the magnetic nanoparticle on the ferromagnetic pattern by applying a first magnetic field to the chemical or biological sensor;
receiving a target substance in the receptor by providing the target substance to the chemical or biological sensor;
detecting the target substance by the chemical or biological sensor using an optical method or an electrical signal measurement method; and
releasing the magnetic nanoparticle and the receptor by applying a second magnetic field which is in a direction opposite to the first magnetic field to the chemical or biological sensor.
